(19) United States
(12) Patent Application Publication
(10) Pub. No.: US 2023/0363691 A1
(43) Pub. Date: Nov. 16, 2023

(54) Ambulatory Brain Monitoring System and Method

(71) Applicant: Sensoria Therapeutics, Inc., Irvine, CA (US)

(72) Inventor: Ross Tsukashima, Irvine, CA (US)

(73) Assignee: Sensoria Therapeutics, Inc., Irvine, CA (US)

(21) Appl. No.: 17/741,243

(22) Filed: May 10, 2022

(51) Int. Cl. A61B 5/384; A61B 5/293; A61B 5/00

(52) U.S. Cl. CPC A61B 5/384; A61B 5/293; A61B 5/4094; A61B 5/4082; A61B 5/0006; A61B 5/002; A61B 5/0031; A61B 5/6864; A61B 2560/0219

(57) ABSTRACT

The devices and methods described below provide for more convenient stereo-electro-encephalography, which may allow the patient to move freely during the days-long monitoring period. The system includes a number of depth SEEG electrodes. In one version of the system, each SEEG electrodes are wirelessly connected to an EEG console, through a subcutaneous electrode which is connected to the SEEG electrode through a conductor. The subcutaneous electrode is in turn wirelessly connected to a supra-cutaneous appliance operable to obtain SEEG signals, generated by the SEEG electrode, through the subcutaneous electrode. The method of use entails implantation of the depth SEEG electrodes deep in the brain, implantation of the subcutaneous electrodes under the scalp. The patient need not be physically connected to a console or control system, and may be ambulatory for the SEEG protocol period.

### FIELD OF THE INVENTIONS

The inventions described below relate to the field of brain monitoring systems for diagnosis and prognosis of epilepsy and other motor disorders.

### BACKGROUND OF THE INVENTIONS

About 1 percent of people suffer from epilepsy, and about a third of those people do not respond to medicine and might only be treated by removal of some part of the brain. Stereo-electro-encephalography (SEEG) is a method for determining whether a patient with epilepsy which has not responded to medication might have “focal epilepsy” which might be treated with brain surgery. Stereo-elector-encephalography requires short term implantation of many electrodes into the brain, in many parts of the brain, and recording of electrical activity detected by the brain in hopes of identifying an area of the brain which is the focus of the epilepsy, which is referred to as an “epileptogenic” area. If an epileptogenic area is identified after analysis of electrical activity, this indicates that the surgical resection of the epileptogenic area might result in alleviation of epileptic seizures. For example, if analysis of the electrical signals from the electrodes indicates that activity in the temporal lobe is the origin of seizures, removal of the temporal lobe may eliminate seizures altogether.

Currently, stereo-electro-encephalography is accomplished by implanting numerous electrodes deep in the brain, with electrical leads extending through the brain, through burr-holes in the skull, and then a bundle of the leads extend several feet to a console or EEG monitoring system, such as a Kohden Neurofax EEG-1200 console, which records data from the leads. The leads have to remain in place for several days (ten to thirty days, typically) to detect a sufficient number of seizures and collect enough data to confirm that an epileptogenic focus has been identified with enough certainty to justify removal or ablation of a part of the patient's brain. After data has been collected, the electrodes are removed. If analysis of the collected electro-encephalography data leads to a conclusion that resection of the “epileptogenic” area will likely lead to cessation of seizures, resection can be performed at any time after removal of the electrodes.

### SUMMARY

The devices and methods described below provide for more convenient stereo-electro-encephalography, which may allow the patient to move freely during the days-long monitoring period. The system and method also entails a lower risk of infection, more discrete usage of the system, and a lower risk of lead breakage and resultant loss of signal, and lower risk of interference because the lead length is much shorter. The system includes a number of SEEG electrodes, which are configured for implantation and explantation in the brain, and configured for use in encephalography. In one version of the system, each SEEG electrode is wirelessly connected to an EEG console, through a subcutaneous electrode which is connected to the SEEG electrode through a conductor. The subcutaneous electrode is in turn wirelessly connected to a supra-cutaneous appliance operable to obtain SEEG signals, generated by the SEEG electrode, through the subcutaneous electrode. Other wireless communication schemes may also be used. The method of use entails implantation of the SEEG electrodes deep in the brain and implantation of the subcutaneous electrodes under the scalp (with the conductor running through the brain). The patient need not be physically connected to a console or control system, and may be ambulatory for the SEEG protocol period.

### BRIEF DESCRIPTION OF THE DRAWINGS

FIG. 1 illustrates a system for treatment of movement disorders using sensor probes.

FIG. 2 illustrates the SEEG electrode, subcutaneous electrode, and patch electrode of FIG. 1.

FIG. 3 illustrates a system similar to the system of FIG. 1 with the addition of an NFC/RFID transponder.

FIG. 4 depicts the system similar to the system of FIG. 1 with the addition of a number of NFC/RFID transponders.

FIG. 5 illustrates an assembly of a SEEG electrode, inductive power supply and NFC/RFID transponder.

FIG. 6 illustrates a SEEG system similar to the system shown in FIG. 4, in which the SEEG electrodes are powered with batteries.

FIG. 7 illustrates an assembly of a SEEG electrode, battery power supply, and NFC/RFID transponder.

FIG. 8 illustrates a SEEG system in which the SEEG electrodes are packaged along with the NFC/RFID components of FIG. 5 or 7 in a single housing.

FIG. 9 illustrates a combined assembly of the NFC/RFID components of FIG. 5 or 7 in a single housing.

### DETAILED DESCRIPTION OF THE INVENTIONS

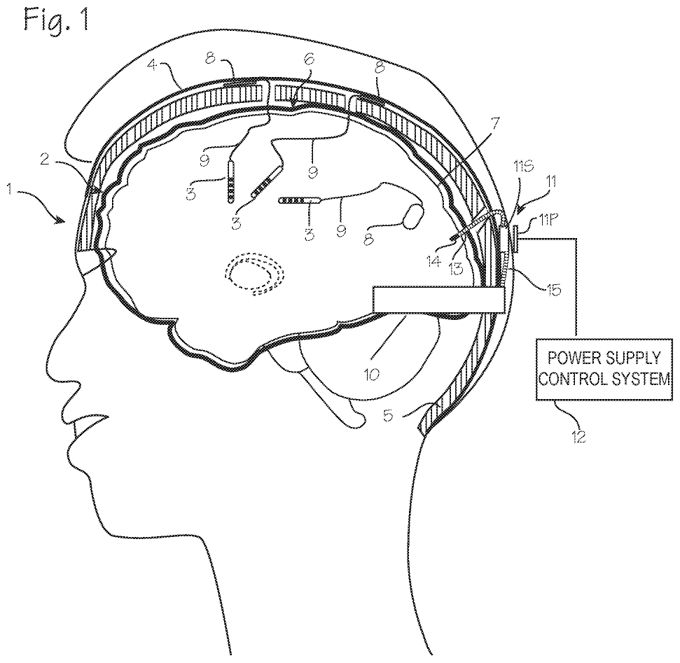

FIG. 1 illustrates a system for monitoring and diagnosis of movement disorders using sensor probes installed in the brain. A patient 1 with a movement disorder and requiring diagnosis of a condition of the brain 2 is illustrated. FIG. 1 shows the placement of a plurality of probes 3. The probes are preferably SEEG electrodes, and may be SEEG “depth electrodes” specifically suited for SEEG protocols and temporary implantation and short-term explantation (but may be other sensor probes and may have stimulation capability). The sensor probes can be inserted entirely within the brain, at various locations in the brain. FIG. 1 also illustrates the scalp 4, the skull 5 which is beneath the scalp, the dura 6 which is beneath the skull and the cerebral cortex 7 which is beneath the dura.

The probes 3 of FIG. 1 are attached to subcutaneous electrodes 8, which are implanted in a subcutaneous location (under the skin, superficial to the skull) under the scalp, through conductors 9, and together these components comprise a SEEG electrode assembly. The probes have been inserted into the brain of the patient through openings (typically, burr-holes) in the skull, and driven through the brain and deposited at a location determined by a surgeon and known to effect target disorders, or known to produce signals indicative of target disorders, such as epilepsy. The leads may be secured in the burr hole with lead anchors to prevent migration during the SEEG monitoring protocol period.

In a first embodiment of the system and method, the system can be configured as described in our co-pending U.S. patent application Ser. No. 17/741,205, filed May 10, 2022, entitled Deep Brain Stimulation System with Wireless Power, the entirety of which is hereby incorporated by reference, and the electrodes may be powered and signals indicative of brain activity (EEG's, for example) obtained through the system disclosed therein. In this first system:

To establish a power circuit from a power source to the probes, and/or communicate sensor data from the probes to a control system or console, in conjunction with the electrodes 8, the patch electrode 10 is disposed on the scalp, supracutaneously on the scalp, or subcutaneously under the scalp. The patch electrode is, preferably, located such that it is not in direct physical contact with the electrodes 8, and is spaced from the electrodes 8. The patch electrode 10 is connected to the secondary (remote) coupling component 11S of a coupling assembly 11, and the primary (base) coupling component 11P is connected to the power supply and control system 12. A conductor 13 extends from the secondary (remote) coupling component 11S, through a burr-hole and into the brain, and may be an insulated wire and include an electrode 14 at its distal end (a conductive wire, insulated or bare, will suffice). An additional conductor connects the secondary (remote) coupling component 11S to the patch electrode 10. The control system is configured to provide power to the probes, for stimulation of brain tissue proximate the probes, and receive sensor data from the probe. The control system or console used to control the system and/or receive and store EEG data from the SEEG electrodes may be implemented on a dedicated console such as the Kohden Neurofax EEG-1200 console (without all the wires), and EEG monitoring system, a general purpose computer, or a mobile phone or tablet.

Preferably, the coupling assembly is an inductive coupling assembly, comprising a pair of coils. The secondary (remote) coupling component 11S may, like the patch electrode, also be installed on the scalp, supra-cutaneously, or subcutaneously under the scalp. The primary (base) coupling component 11P may be placed in proximity to the secondary coupling using a magnetic attachment or other releasable attachment means (a snap lock fitting, secured to a headband, glued to the overlying skin with a weak adhesive) or non-releasable means (stitched to the scalp, nailed or screwed to the skull, or other means not considered “releasable” attachment means which require tools for removal).

The control system 12 is operable to generate power and transmit power through the inductive coupling to the electrodes and may be further programmed to analyze the sensor data and modify control signals to the probes to control the stimulation provided by the probes in response to the sensor data. The power supply and control system 12 may be disposed in an appliance, which may be configured as headwear (disguised as a hat, head band, or wig). The power supply may be connected to the SEEG electrodes through the inductive coupling, and may be connected substantially continuously during the SEEG monitoring protocol period of several days to several weeks. The power supply is preferably not implanted subcutaneously on the patient's chest of abdomen, as is the practice with pulse generators used with DBS systems.

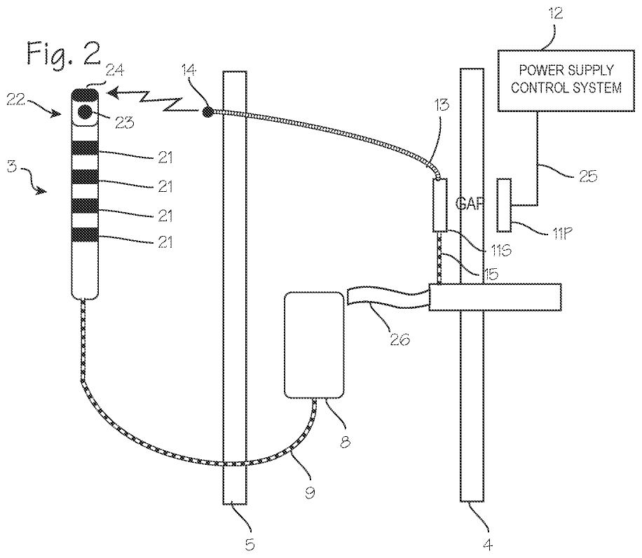

FIG. 2 illustrates the SEEG electrode 3, subcutaneous electrode 8, and patch electrode 10 of FIG. 1 and the circuit for obtaining EEG signals from the probe. The probe 3 includes several sensor/stimulation electrodes 21, and an LED assembly 22 which includes an LED 23, and a power contact 24 for providing power to the electrodes 21 and the LED 23 or providing a ground path/return path for the LED. The sensor/stimulation electrodes are connected to the subcutaneous electrode 8, and may be operated, in conjunction with the subcutaneous electrode 8 to apply electrical stimulation to brain tissue or sense electrical signals of the brain, or both. FIG. 2 also shows the conductor 9 and subcutaneous electrode 8. The subcutaneous electrode 8, which is preferably configured for implantation under the scalp (in the subcutaneous tissue 25 of the scalp (which may include connective tissue, epicranial apaneurosis, areolar connective tissue, and periosteum)), may consist of a bare conductive plate (metal such as stainless steel, or carbon fiber or glassy/pyrolytic carbon), but may also consist of a conductive plate covered in a thin layer of insulation. The subcutaneous electrode 8 may also comprise a near field communication coil (an NFC/RFID tag) configured to communicate with the control system through a corresponding emitter and NFC/RFID controller of the control system (which may be housed proximate the patch electrode or housed in a separate appliance). The conductor 9 also functions as a tether for retrieval of the probe from the brain. The power contact 24 on the probe tip may be bare, uncoated metal or other conductive material, or may be coated with a non-insulating coating with or without biologic effect, or covered in an insulating layer and work in conjunction with an insulated subcutaneous electrode 8 for capacitive coupling of both components of the probe to the conductor 13 of the secondary coil 11S. The conductors 9 may be floppy, having little or no columnar strength, with a small diameter merely sufficient to conduct signals to the subcutaneous electrodes. Thus, the conductors may be characterized by column strength insufficient to support pushing the probe through the brain, and the conductors may or may not include a lumen which accommodates a stylet which is used in some depth electrodes to stiffen the length of the SEEG electrode in order to push it through the brain.

The system may be used in a method in which a surgeon implants a number of SEEG electrodes 3 (about 20) in the brain of a patient, in areas of the brain which may be the focus or origin of brain activity associated with a movement disorder, and places the subcutaneous electrodes under the scalp of the patient, in the subcutaneous tissue 25 of the scalp (which may include connective tissue, epicranial apaneurosis, areolar connective tissue, and periosteum), and implanting the patch electrode also subcutaneously, and implanting the secondary (remote) coupling component 11S pericutaneously (preferably subcutaneously, or perhaps supra-cutaneously), and installing the tip of the conductor 13 in the brain. After installation of these components, the surgeon or the patient may place the primary (base) coupling component 11P proximate the secondary (remote) coupling component 11S. With the primary (base) coupling component 11P coupled to the secondary (remote) coupling component 11S, the control system may be operated to collect EEG signals from the various implanted SEEG electrodes. The coupling may be maintained for the entire monitoring period, for substantially the entire period (excepting brief periods). EEG data may be collected continuously while the coupling is maintained.

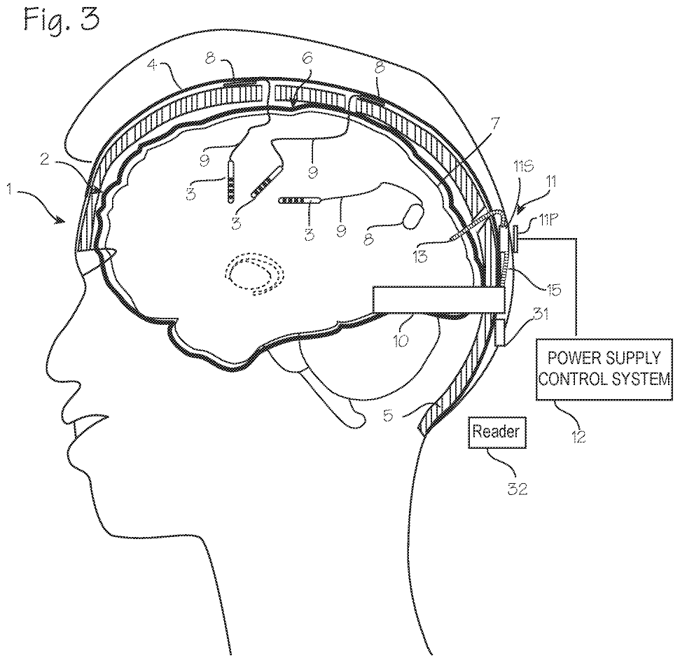

An embodiment which includes memory is depicted in FIG. 3, which depicts the system similar to the system of FIG. 1 with the addition of an NFC/RFID transponder. The system of FIG. 1 may be modified by adding memory for storage of data, so that the system may be operated continuously to collect EEG data but need not be continuously connected to the control system. The system may include a single NFC/RFID transponder assembly 31 (a tag) which may be located in any convenient location but is shown in a peri-cutaneous location (subcutaneous or supra-cutaneous proximate the secondary (remote) coupling component 11S), and may be formed integrally with the secondary (remote) coupling component 11S. This NFC/RFID transponder 31 is electrically connected to the patch electrode for collecting EEG data, and may be electrically connected to the secondary (remote) coupling component 11S for power. The NFC/RFID transponder 31 includes memory for storing EEG data from the several SEEG electrodes 8. In this particular system, the SEEG electrodes 8 and NFC/RFID transponder 31 may be continuously powered through the inductive coupling, through a power source disposed on the scalp or more remotely (outside the brain). The system includes an NFC/RFID reader 32, which is operable to occasionally interrogate the NFC/RFID transponders 31 and obtain EEG data from the SEEG electrodes which has been stored in memory on the NFC/RFID transponder 31. The NFC/RFID reader 32 may store the data and/or transmit it to a control system for analysis of the EEG by a neurologist.

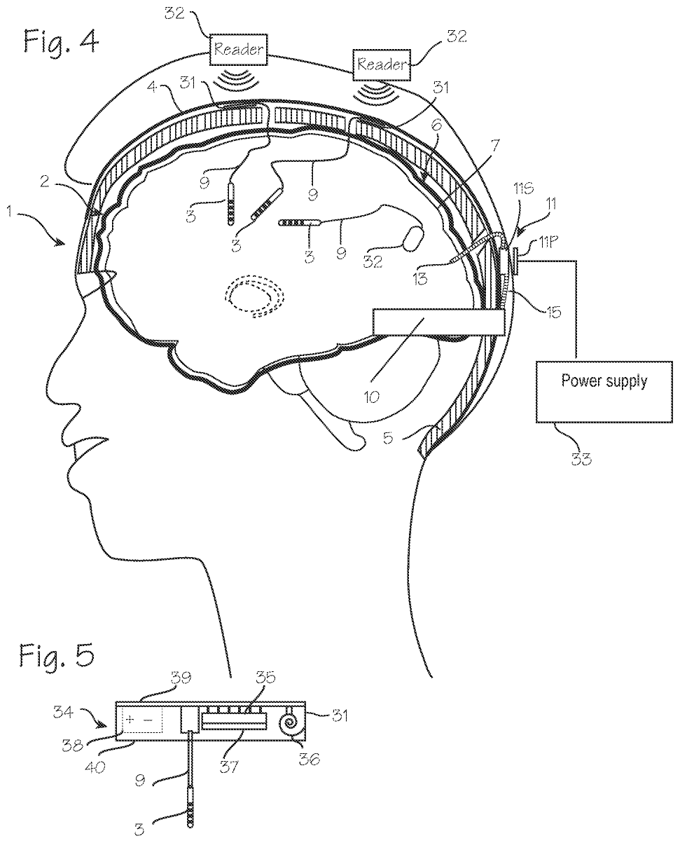

Another variation of the system which includes memory is depicted in FIG. 4, which depicts the system similar to the system of FIG. 1 with the addition of a number of NFC/RFID transponders. Each subcutaneous electrode 8 may be replaced with an NFC/RFID transponder 31, with memory for storing historical EEG data. The NFC or RFID transponders 31 in this system are preferably powered in the same manner as the SEEG sensors, with power supplied from a power supply 33 through the inductive coupling 11. The NFC or RFID transponders 31 may instead include a small power source, or a radio chip attached to an antenna for wireless power needed to operate the SEEG electrodes and store EEG data. This system includes an NFC/RFID reader 32 (several readers, or a single reader) which are operable to occasionally interrogate the NFC/RFID transponders 31 and obtain EEG data from the SEEG electrodes which has been stored on each NFC/RFID transponder 31. The NFC/RFID reader(s) 32 may store the data and/or transmit it to a control system for analysis of the EEG by a neurologist.

FIG. 5 illustrates a transponder and SEEG electrode assembly 34 which includes the NFC/RFID transponder 31, a SEEG electrode 3 and NFC/RFID transponder microchip 35, NFC/RFID antenna 36 and memory 37. A battery 38 may be included. These components may be mounted on circuit board 39 and housed in a housing 40.

The transponder microchip 35 may be powered through the inductive power coupling of the circuit of FIG. 4, and a battery need not be included. The microchip and memory may also be operable with or without a broadcast electric field (as in FIG. 8).

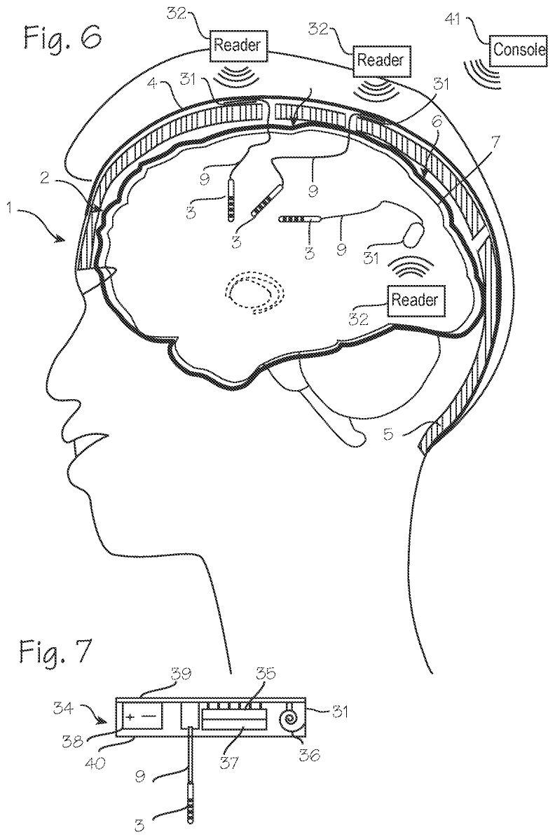

FIG. 6 illustrates a SEEG system similar to the system shown in FIG. 4, in which the SEEG electrodes are powered with batteries instead of the inductive coupling shown in FIG. 4. The system of FIG. 6 includes the SEEG electrodes 3, the conductors 9, and NFC/RFID transponder 31, but does not include the inductive power coupling 11, patch electrode 10, or subcutaneous electrodes 8 used to power the system of earlier figures. Instead, this system may be powered by batteries held in the transponder assembly 31.

FIG. 7 illustrates an assembly of the NFC/RFID transponder 31 and a SEEG electrode 3 and NFC/RFID transponder microchip 35, NFC/RFID antenna 36 and memory 37. This assembly includes a battery power supply 38 which powers the various components. This system may use a separate NFC/RFID reader 32 for each NFC/RFID transponder 31, or it may use a single NFC/RFID transponder 31 operable to interrogate and obtain EEG data from several NFC/RFID transponders 31. In this system, the NFC/RFID transponder is configured to store EEG data for transmission to a console or computer 41 which is configured to receive and store the EEG data, but is not configured to control the SEEG electrodes.

In use, the system of FIGS. 6 and 7 may be used in a method in which a surgeon implants a number of SEEG electrodes 3 (about 20) in the brain of a patient, in areas of the brain which may be the focus or origin of brain activity associated with a movement disorder, and places the NFC/RFID transponders 31 under the scalp of the patient, in the subcutaneous tissue 25 of the scalp (which may include connective tissue, epicranial apaneurosis, areolar connective tissue, and periosteum),

After installation of these components, the surgeon or the patient may place an NFC/RFID reader 32 proximate an NFC/RFID transponder 31 to interrogate the transponders and obtain EEG data stored in the memory of the transponder. The surgeon or the patient may use a different reader for each transponder, or may use a single reader to interrogate several, or all, of the transponders. The readers may be maintained proximate the transponders, for substantially the entire period, and EEG data may be collected continuously while the coupling is maintained. However, because the transponders are self-powered and are operable to store historical EEG data, the surgeon or patient may bring the readers into proximity with the transponders only periodically, and the EEG data may be collected only periodically. This leaves the patient free to engage in normal activity, interrupted occasionally to interrogate the transponders, without the need for constant use of an appliance or head wear to keep the reader installed on the head in proximity to the transponders.

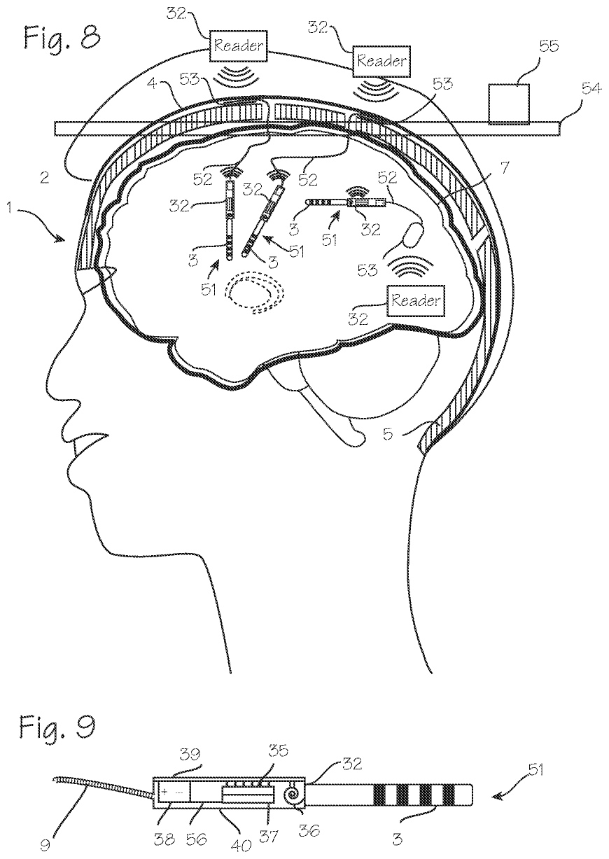

FIG. 8 illustrates a SEEG system in which the SEEG electrodes are packaged along with the NFC/RFID components of FIG. 5 or 7 in a single housing, and this combined electrode/transponder is secured to a tether not used for transmission of power or data and used only for retrieval of the combined electrode/transponder at the completion of the SEEG protocol. The system of FIG. 9 includes combined SEEG electrodes 3 and NFC/RFID transponders 31 in a combined assembly 51. The tether 52 may be electrically non-functional, and may serve only to facilitate removal of the combined assembly from the brain after completion of a SEEG protocol. Likewise, tabs 53 may be electrically non-functional, and may serve only to facilitate secure placement of the end of the tether under the scalp (preventing migration through the insertion burr-hole, for example) and to facilitate grasping of the ends of the tethers for removal of the combined assembly from the brain after completion of a SEEG protocol. The tab may be omitted, and the end of the tether, unattached to any additional structure, may suffice as a graspable feature sufficient to retrieve the SEEG electrode assembly after the SEEG protocol is complete. This system may use a power transmitting antenna 54 to power the electrodes and transponder components. The antenna may also be configured as an NFC/RFID reader in place of readers 32. If the hoop antenna is used, the control system 55 operable to transmit wireless power through the antenna to the electrode and transponder, may be used to control power applied through the hoop to the electrodes and transponder components. The loop antenna and control system may be used in place of a reader to obtain sensor data from the electrodes.

As with previously described systems, this system may include memory and battery power disposed on the transponder, so that the reader may be used occasionally to collect EEG data collected over an extended period of time. The transponder may be configured without an onboard power supply, and the antenna may be applied and operated continuously to provide power to the NFC/RFID transponder 31, and the transformer may be configured without onboard memory, and the antenna used continuously to collect ECG data from the transponder.

FIG. 9 illustrates the combined assembly 51 of the NFC/RFID transponder 31 and a SEEG electrode 3 and NFC/RFID transponder microchip 35, NFC/RFID antenna 36 for use in the system shown in FIG. 8. As with the previously describe systems, this assembly may include a battery power supply 38 which powers the various components and memory 37 for storage of EEG data, in which case the data may be collected by occasional operation of the NFC/RFID reader 32 without the need to continuously wear and use the NFC/RFID reader 32. Also as with previously described systems, the battery may be omitted and the combined assembly of the transponder may include a power converter 56 and the electrodes, transponder and memory may be powered by the power converter. In this system, the power converter is powered wirelessly from the antenna external to the skull, disposed in an appliance or disguised as headwear, which is worn continuously while collecting EEG data (this may minimize the size of the implanted components but require continuous application of the appliance/headwear). This system may use a separate NFC/RFID reader 32 for each NFC/RFID transponder 31, or it may use a single NFC/RFID transponder 31 operable to interrogate and obtain EEG data from several NFC/RFID transponders 31.

The method of obtaining EEG data from a patient's brain described above uses several of the SEEG electrode assemblies which each comprise a SEEG electrode 3 secured to a retrieval tether (9, 52) having a first end and a second end, with the SEEG electrode 8 secured to the first end of the tether. The method includes the steps of providing a plurality of SEEG electrode assemblies (3, 9, 8; 9, 31, 52, 53) for temporary implantation into the brain, for the length of a SEEG protocol, which may be several days or weeks, and, preferably, removing the electrodes after sufficient data has been collected to diagnose a patient's condition, (2) providing a control system configured to collect and store EEG data obtained from the SEEG electrodes, (3) for each of the SEEG electrode assemblies, implanting the SEEG electrode in the patient's brain by inserting the SEEG electrode through a burr hole, with the tether running from the SEEG electrode and through the burr hole and securing the second end of the tether subcutaneously or supracutaneously outside the skull of the patient; (4) wirelessly communicating EEG data obtained from the SEEG electrodes to the control system, without connecting the SEEG electrodes to the control system with wires; and (5) after collecting EEG data from the SEEG electrodes, using the tether to remove the SEEG electrodes from the patient's brain. In this method, the SEEG electrodes are implanted in the brain in a location known to effect target disorders, or known to produce signals indicative of target movement disorders, such as epilepsy. With this method, doctors can collect EEG data and determine if the movement disorders are likely treatable with procedures such as resection of portions of the brain, stimulation of certain areas of the brain, or administration of drugs to the patient.

In one version of the method, the SEEG electrode assembly may include an NFC/RFID transponder 31 fixed to the second end of the tether, and the method may include implanting the NFC/RFID transponder 31 pericutaneously, under or on the patient's scalp and wirelessly collecting EEG data from the SEEG electrodes using an NFC/RFID reader. After collecting EEG data from the SEEG electrodes, the method preferably includes removing the NFC/RFID transponder 31 from the patient's scalp, along the removing the SEEG electrodes from the brain (although the electrodes may be left in the brain for later EEG acquisition and administration of stimulation to the brain).

In another version of the method, each SEEG electrode assembly may also comprise an electrically non-functional tab 53 fixed to the second end of the tether. This version of the method further comprises the steps of implanting the electrically non-functional tab 53 pericutaneously, under or on the patient's scalp and wirelessly collecting EEG data from the SEEG electrodes using an NFC/RFID reader, and, after collecting EEG data from the SEEG electrodes, pulling the electrically non-functional tab 53 to remove the SEEG electrode from the patient's brain.

In another version of the method, each SEEG electrode assembly may also comprises an electrode 8 fixed to the second end of the tether, wherein the tether comprises an electrical conductor, and the system includes at least one NFC/RFID transponder operable to transmit EEG data from the probes to an NFC/RFID reader. This version of the method entails implanting the electrode 8 pericutaneously, under or on the patient's scalp, wirelessly collecting EEG data from the SEEG electrodes using an NFC/RFID reader, and, after collecting EEG data from the SEEG electrodes, pulling the electrode 8 to remove the SEEG electrode from the patient's brain.

Each of the versions of the system and methods can include means for wirelessly powering the SEEG electrodes, or a battery operably connected to the SEEG electrode, and any of the several powering systems and methods may be used with any of the EEG data sensing systems and methods.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. A method of obtaining EEG data from a patient's brain, said method comprising the steps of:
providing a plurality of SEEG electrode assemblies (3, 9), each said SEEG electrode assembly comprising a SEEG electrode (3) secured to a retrieval tether (9) having a first end and a second end, said SEEG electrode (3) secured to the first end of the tether;
providing a control system (12) configured to collect and store EEG data obtained from the SEEG electrode (3);
for each of the plurality of the SEEG electrode assemblies, implanting the SEEG electrode (3) in the patient's brain by inserting the SEEG electrode (3) through a burr hole, with the retrieval tether (9) running from the SEEG electrode and through the burr hole;
securing the second end of the tether subcutaneously or supracutaneously outside the skull of the patient;
wirelessly communicating EEG data obtained from the SEEG electrodes (3) to the control system, without connecting the SEEG electrodes (3) to the control system (12) with wires;
after collecting EEG data from the SEEG electrodes, using the retrieval tether (9) to remove the SEEG electrodes (3) from the patient's brain.

2. The method of claim 1, further comprising the steps of:
implanting each SEEG electrode (3) in the brain in a location known to effect target disorders, or known to produce signals indicative of target disorders.

3. The method of claim 2, wherein:
the target disorder is a movement disorder.

4. The method of claim 2, wherein:
the target disorder is epilepsy.

5. The method of claim 1, wherein each SEEG electrode (3) further comprises an NFC/RFID transponder (31) fixed to the second end of the tether (9), and wherein the method further comprises the steps of:
implanting the NFC/RFID transponder (31) pericutaneously, under or on the patient's scalp;
wirelessly collecting EEG data from the SEEG electrodes using an NFC/RFID reader (32);
after collecting EEG data from the SEEG electrodes, removing the NFC/RFID transponder (31) from the patient's scalp.

6. The method of claim 1, wherein each SEEG electrode (3) further comprises an electrically non-functional tab (53) fixed to the second end of the tether (9), and wherein the method further comprises the steps of:
implanting the electrically non-functional tab (53) pericutaneously, under or on the patient's scalp;
wirelessly collecting EEG data from the SEEG electrodes (3) using an NFC/RFID reader (32);
after collecting EEG data from each SEEG electrode (3), pulling the electrically non-functional tab (53) to remove each SEEG electrode (3) from the patient's brain.

7. The method of claim 1, wherein each SEEG electrode (3) further comprises an electrode (8) fixed to the second end of the tether (9), wherein the tether comprises an electrical conductor, and wherein the method further comprises the steps of:
implanting the electrode (8) pericutaneously, under or on the patient's scalp;
wirelessly collecting EEG data from the SEEG electrodes using an NFC/RFID reader (32);
after collecting EEG data from the SEEG electrodes (3), pulling the electrode (8) to remove the SEEG electrode (3) from the patient's brain.

8. The method of claim 5, 6 or 7, further comprising the step of:
wirelessly powering the SEEG electrodes (3).

9. The method of claim 5, 6 or 7, further comprising the step of:
powering the SEEG electrodes (3) with a battery (38) operably connected to the SEEG electrode (3).

10. The method of claim 8, wherein the step of wirelessly powering the SEEG electrodes comprises the step of:
wirelessly powering each SEEG electrode (3) using an inductive coupling (11).

11. The method of claim 10, further comprising the step of:
wirelessly powering the SEEG electrodes using an inductive coupling (11) by:
affixing a secondary (remote) coupling component (11S) of an inductive coupling assembly (11) comprising a secondary (remote) coupling component (11S) and primary (base) coupling component (11P) to the scalp of the patient, peri-cutanously, and placing the primary (base) coupling component (11P) proximate the secondary (remote) coupling component (11S);
implanting a patch electrode subcutaneously between the scalp and skull of the patient;
electrically connecting the secondary (remote) coupling component (11S) to the brain of the patient through a second electrical conductor;
electrically connecting the secondary (remote) coupling component (11S) to the patch electrode through a third electrical conductor;
connecting a power supply to the primary (base) coupling component (11P); and
operating the power supply to power to the SEEG electrode, through a circuit established from the secondary (remote) coupling component (11S), through the second electrical connector (15), through brain tissue to a power contact on the SEEG electrode (3), through the first electrical conductor to the subcutaneous electrode (8), through scalp tissue to the patch electrode, and through the third electrical conductor to the secondary (remote) coupling component (11S).

12. The method of claim 8, wherein:
the SEEG electrode further comprises a power converter (12), operable to receive power from a power transmitting antenna;
and the step of wirelessly powering the SEEG electrodes comprises the step of:
providing wirelessly powering the SEEG electrodes using an inductive coupling (11).

13. The method of claim 10, further comprising the step of:
obtaining SEEG signals from each of the plurality of the SEEG electrodes through the inductive coupling (11).

14. The method of claim 5, further comprising the step of:
powering the SEEG electrodes with a battery (38) operably connected to the SEEG electrode with the steps of:
providing the NFC/RFID transponder (31) with a battery connected to the SEEG electrode through the tether (9), wherein the tether is electrically conductive.

15. The method of claim 5, further comprising the step of:
powering the SEEG electrodes with a battery operably connected to the SEEG electrode with the steps of:
providing the NFC/RFID transponder with a battery connected to the SEEG electrode through the tether, wherein the tether is electrically conductive.

16. A system for obtaining EEG data from a patient's brain, said method comprising the steps of:
a plurality of SEEG electrode assemblies (3, 9, 8; 3, 9, 31, 3, 52, 53), each said SEEG electrode assembly comprising:
a SEEG electrode (3) secured to a retrieval tether (9, 52) having a first end and a second end, said SEEG electrode (3) secured to the first end of the tether;
a retrieval means (8, 31, 52) fixed to the second end of the tether, said retrieval means configured for temporary peri-cutaneous implantation in a scalp of the patient;
a control system (12) configured to collect and store EEG data obtained from the SEEG electrodes;
means for wirelessly communicating EEG data from the SEEG electrodes to the control system.

17. A system of claim 16, further comprising means for wirelessly powering the SEEG electrodes (3).

18. The system of claim 16, wherein the system does not include electrical wires communicating from the SEEG electrode (3) to the control system (12).

19. The system of claim 16, wherein:
the tether is electrically conductive; and
the retrieval means further comprises an electrode configured for percutaneous placement on the patient; and
the system further comprises an inductive power coupling operable to supply power to the SEEG electrodes through the electrode.

20. The system of claim 16, wherein:
the tether 52 is electrically non-functional; and
the retrieval means further comprises an electrically non-functional tab 53 configured for percutaneous placement on the scalp of the patient; and
an external transmitter 54 operable to power the SEEG electrode;
an NFC/RFID reader 32; and
the SEEG electrode further comprises a power converter, said power converter operable to convert radiofrequency energy from the external transmitter to power the SEEG electrode, and an NFC/RFID transponder microchip 35 operable to transmit EEG data to the NFC/RFID reader (32).

21. A system of claim 16, wherein:
the tether is electrically functional; and
the retrieval means comprises an NFC/RFID transponder (31) connected to the SEEG electrode through the electrically conductive tether, said NFC/RFID transponder operable to transmit EEG data from the SEEG electrodes to an NFC/RFID reader (32).
